(19) United States
(12) Patent Application Publication
(10) Pub. No.: US 2015/0208645 A1
(43) Pub. Date: Jul. 30, 2015

(54) PHYTOSANITARY COMPOSITIONS COMPRISING AN ETHER-AMIDE COMPOUND

(71) Applicant: RHODIA OPERATIONS, Paris (FR)

(72) Inventors: Thierry Vidal, Lyon (FR); Valerio Bramati, Arese (IT)

(73) Assignee: RHODIA OPERATIONS, Paris (FR)

(21) Appl. No.: 14/416,926

(22) PCT Filed: Jul. 25, 2013
(86) PCT No.: PCT/EP2013/065757

§ 371 Date: Jan. 23, 2015

(30) Foreign Application Priority Data
Jul. 26, 2012 (FR) 12 57272

(51) Int. Cl. A01N 25/02; C07C 235/06; A01N 25/30

(52) U.S. Cl. CPC A01N 25/02; A01N 25/30; C07C 235/06

(57) ABSTRACT

The object of the present invention is phytosanitary compositions comprising an active phytosanitary product and an ether-amide compound. The ether-amide compound may natively be present as a solvent, co-solvent, crystallization inhibitor or an agent for increasing bioactivity of the active phytosanitary product.

The present invention relates to phytosanitary compositions comprising an active phytosanitary product and an ether-amide compound. The ether-amide compound may notably be present as a solvent, co-solvent, crystallisation inhibitor or agent for increasing the bioactivity of the active phytosanitary product.

Agriculture uses many active materials as fertilizers or pesticides, for example insecticides, herbicides or fungicides. These are referred to as active phytosanitary products (or of active material). Active phytosanitary products are generally produced in a pure or highly concentrated form. They however have to be used on farms at low concentrations. For this purpose, they are generally formulated with other ingredients in order to allow easy weight dilution by the farmer. These are referred to as phytosanitary formulations. The dilution carried out by the farmer is generally achieved by mixing the phytosanitary formulation with water.

Thus, phytosanitary formulations have to allow easy dilution by weight by the farmer, in order to obtain a product in which the phytosanitary product is properly dispersed, for example as a solution, emulsion, suspension, or suspo-emulsion. For example, solvents may be used for formulating active phytosanitary products, for example in the form of emulsifiable concentrates (Emulsifiable Concentrate “EC”) intended to be diluted in water by the farmer, before application on a field.

Phytosanitary formulations thus allow the transport of a phytosanitary product in a relatively concentrated form, easy packaging and/or easy handling for the final user.

Certain solid phytosanitary active ingredients are often difficult to formulate. This is for example the case of tebuconazole, which is a particularly efficient fungicide, and of widespread use, notably for soya bean cultivation. For certain phytosanitary active ingredients, it is difficult to produce concentrated formulations, easy to be diluted for the farmer, stable and without any substantial drawbacks (either found or perceived) in terms of safety, toxicity and/or echo-toxicity. For certain active ingredients, it is difficult to formulate them with relatively high concentrations, with sufficient stability. In particular, it is necessary to avoid the occurrence of crystals in particular at a low temperature and/or during dilution and/or during storage at a high temperature of the diluted composition. The crystals may have negative effects, notably clogging filters of the devices used for spreading out the diluted composition, clogging the spraying devices, reducing the overall activity of the formulation, generating unnecessary problems in waste streams for removing the crystals, and/or cause a poor distribution of the active product on the agricultural field.

The industry is searching for compounds which may have solvent properties and may allow variation or optimisation of phytosanitary formulations. The industry notably needs compounds of moderate cost, having interesting properties of use. The industry also needs compounds having a toxicological and/or ecotoxicological profile perceived as favourable, notably low volatility (low VOC), good biodegradability, low toxicity and/or low hazard.

The use of dialkylamides as solvents is known. These are products of formula R—CONMe₂ wherein R is a hydrocarbon group such as an alkyl, typically a C₆-C₃₀ alkyl. Such products are notably marketed under the name of Genagen® by Clariant. These solvents find applications notably in the phytosanitary field. These solvents, however, have a restricted field of use and do not allow solubilization of certain phytosanitary active ingredients at certain concentrations, in useful temperature ranges, without formation of crystals. Moreover they are generated from an expensive raw material.

A need remains for novel phytosanitary formulations, in particular phytosanitary formulations having relatively high concentrations of active phytosanitary products, which may have good stability, for example by reducing, or even preventing the formation of crystals notably at a low temperature and/or during dilution and/or during storage at a high temperature of the diluted composition.

The present invention aims at addressing at least one of the needs expressed above and it manages this by applying a particular ether-amide compound.

Thus, the present invention according to one its aspects, relates to a phytosanitary formulation comprising at least:

a) an active phytosanitary product,

b) a compound of the following formula (I):

R^{a}R^{b}C(OR¹)-A-C(O)—NR²R³ (I)

wherein:
R^{a} and R^{b}, either identical or different, are groups selected from the hydrogen atom and linear or branched, notably C₁-C₆ alkyl groups;
R¹ is a hydrocarbon group comprising an average number of carbon atoms ranging from 1 to 36, saturated or unsaturated, linear or branched, optionally cyclic, optionally aromatic, optionally substituted;
R² and R³, identical or different, are groups selected from the hydrogen atom and hydrocarbon groups comprising an average number of carbon atoms ranging from 1 to 36, saturated or unsaturated, linear or branched, optionally cyclic, optionally aromatic, optionally substituted, R² and R³ may optionally form together a ring comprising the nitrogen atom to which they are bound, optionally substituted and/or optionally comprising an additional heteroatom,
A represents a linear or branched alkyl group, the main chain of which comprises at least 2 carbon atoms;

c) optionally a surfactant, and

d) optionally water.

The invention also relates according to another of its aspects, to the use of a compound of formula (I) according to the invention in phytosanitary formulations comprising a), optionally c) and optionally d).

The invention also relates, according to another of its aspects, to the use of a compound of formula (I) according to the invention in phytosanitary formulations comprising a), optionally c) and optionally d), as a solvent, co-solvent, crystallisation inhibitor, and/or agent for increasing the bioactivity of said active phytosanitary product.

The invention also relates according to another of its aspects, to a method for preparing phytosanitary formulations comprising a step for mixing a), b), optionally c), and optionally d).

The invention also relates to the use of a phytosanitary formulation according to the invention for treating agricultural surfaces as well as a method for treating agricultural surfaces comprising at least one step for applying a phytosanitary formulation according to the invention, generally in diluted form.

### DEFINITIONS

Within the scope of the present invention, the intention is to designate by:
“compound of the invention”, a compound of formula (I) according to the invention;
“material composition”, a more or less complex composition comprising several chemical compounds. This may be typically a non-purified or moderately purified reaction product.

For example, the compound of the invention may notably be isolated and/or marketed and/or used as a material composition comprising it. The compound of the invention, in the form of a pure molecule or in the form of a mixture fitting formula (I), may thus be comprised in a material composition in the sense of the invention.

Moreover, within the scope of the present invention, the term of “solvent” is understood in a broad sense, notably covering the functions of a co-solvent, and of a crystallisation inhibitor.

The term of solvent may notably designate a product liquid at the temperature of use, preferably with a melting temperature of less than or equal to 20° C., preferably than 5° C., preferably than 0° C., which may contribute to make a solid material liquid, making a liquid material more fluid, or preventing or delaying solidification or crystallisation of the material in a liquid medium.

Phytosanitary Formulations

In the sense of the invention, a phytosanitary formulation is a phytosanitary formulation, preferably concentrated, comprising at least one active compound.

By <<concentrated>> is meant to designate a formulation for which the weight concentration of the active ingredient is comprised between 10% and 80% based on the total weight of the formulation.

The phytosanitary formulation of the invention is preferably in liquid form.

Different types of phytosanitary formulations may be applied, notably according to the different phytosanitary products. For example mention is made of emulsifiable concentrates (Emulsifiable Concentrates <<EC>>), soluble concentrates (Soluble Concentrate <<SL>>), concentrated emulsions (Emulsion in water “EW”), microemulsions (“ME”), wettable powders (Wettable Powders <<MP>>), granules which are dispersible in water (Water Dispersible Granules, <<WDG>>), suspo-emulsions (“SE”). The formulations may depend on the physical form of the phytosanitary product (for example solid or liquid), and on its physico-chemical properties in the presence of other compounds such as water or non-aqueous dispersion, emulsification or solubilization media.

After weight dilution by the farmer, for example by mixing it with water, the phytosanitary product may be found in different physical forms: solution, dispersion of solid particles, dispersion of droplets of the product, droplets of solvent in which the product is dissolved.

The formulation of the invention may notably exhibit:
solubilization of significant amounts of active ingredients,
lack of crystallisation, even in demanding conditions,
an increase in the biological activity which may be due to good solvation, and/or
a safety, toxicology and/or eco-toxicology profile perceived as favorable.

Details concerning the products a), b), c), and d) are given below.

Compound b) of Formula (I)

The compound b) has the general formula (I) given above. It is noted that this may be mixture of several compounds of general formula (I). In other words, the compound may be alone or in a mixture. Within the scope of mixtures of several compounds, the number of atoms or units may be expressed as an average number. These are number average numbers. In the case of compounds alone, these will be generally integers, as regards the number of carbon atoms.

The groups R^{a} and R^{b}, identical or different, are groups selected from the hydrogen atom and linear or branched alkyl groups. The alkyls may notably be C₁-C₆, preferably C₁-C₃ alkyls. These may notably be methyl or ethyl groups.

According to a particular embodiment, at least one of the groups selected from R^{a} and R^{b} is different from a hydrogen atom, for example a group selected from linear or branched alkyl groups. The alkyls may notably be C₁-C₆, preferably C₁-C₃ alkyls. These may notably be methyl or ethyl groups.

According to particular embodiments:
R^{a}═H and R^{b}═H, or
R^{a}=—CH₃ and R^{b}═H, or
R^{a}=—C₂H₅ and R^{b}═H.

According to a particular embodiment, the total number of carbon atoms within the compound b), except for the groups R¹, R² and R³, is 4, 5, 6, 7 or 8, or average numbers between each of these values.

The group A represents a linear or branched alkyl group, the main chain of which comprises at least 2 carbon atoms.

By <<main chain>> is meant to refer in the sense of the invention to the hydrocarbon chain binding the carbon atom bearing the amide function and the carbon atom bearing the ether function, both of these carbon atoms being excluded.

According to an embodiment, the group A represents a linear or branched alkyl group, the main chain of which comprises at least 2 carbon atoms, for example from 2 to 8 carbon atoms, for example from 2 to 6 carbon atoms, preferably from 2 to 4 carbon atoms.

According to an embodiment, the group A represents a linear alkyl group.

According to another embodiment, the group A represents a branched alkyl group.

In this case, this may be a branched alkyl group with at least one alkyl group, itself linear or branched, comprising from 1 to 10 carbon atoms, for example from 2 to 8 carbon atoms. These may notably be ethyl or methyl groups.

The groups R² and R³, identical or different, are groups selected from a hydrogen atom and hydrocarbon groups comprising an average number of carbon atoms ranging from 1 to 36, saturated or unsaturated, linear or branched, optionally cyclic, optionally aromatic, optionally substituted, R² and R³ may optionally form together a ring comprising the nitrogen atom to which they are bound, optionally substituted and/or optionally comprising an additional heteroatom. It should be noted that R² and R³ are not simultaneously hydrogen atoms. In other words, the group —CONR²R³ is not a group —CONH₂. This may be a group —CONHR² wherein R² is not a hydrogen atom, or a group —CONR²R³ wherein R² and R³ are not hydrogen atoms.

R² and R³, identical or different, may for example be selected from the methyl, ethyl, propyl(n-propyl), isopropyl, n-butyl, isobutyl, n-pentyl, amyl, isoamyl, hexyl, cyclohexyl groups, mixtures and/or associations thereof. R² and R³ may also be such that they form together with the hydrogen atom a morpholine, pyrrolidine, piperazine or piperidine group.

According to a preferred embodiment, R² and R³ are both methyl groups.

The group R¹ is a hydrocarbon group comprising an average number of carbon atoms ranging from 1 to 36, notably from 1 to 25, in particular from 1 to 20, saturated or unsaturated, linear or branched, optionally cyclic, optionally aromatic, optionally substituted.

This may notably be a hydrocarbon group selected from methyl, ethyl, propyl(n-propyl), isopropyl, n-butyl, isobutyl, n-pentyl, amyl, isoamyl, hexyl, or cyclohexyl groups.

According to an embodiment, R1 is selected from a methyl and a methyl group.

According to a particular embodiment R^{a}═R^{b}═H and R¹ is an alkyl group comprising less than 10 carbon atoms, for example less than 5 carbon atoms, for example a methyl or ethyl group.

According to another embodiment R^{a}=—CH₃ or C₂H₅, R^{b}═H and R¹ is an alkyl group comprising less than 10 carbon atoms, for example less than 5 carbon atoms, for example a methyl or ethyl group.

According to an embodiment, the compound of the invention may be totally miscible in water.

According to a particular embodiment, the compound of the invention is partly miscible with water. Miscibility in water may for example be less than 10% by weight (25° C.), preferably 2%, preferably 1% or 0.1%. It mw be greater than 0.001%, preferably 0.01% or 0.1%. For example it may be comprised between 0.01% and 2%, for example between 0.1% and 1%.

Surprisingly, the compounds of the invention have good solvent properties, notably for phytosanitary active ingredients in phytosanitary formulations with low miscibility in water. The groups R^{a}, R^{b}, A and/or the group R¹ and/or the groups R², R³ may be selected so as to control miscibility in water. In the case when the compound is miscible with water, it is possible to advantageously associate the compound of formula (I) with a co-solvent. Details as regards the co-solvents are given below.

The compound of the invention may notably have one of the following formulae:

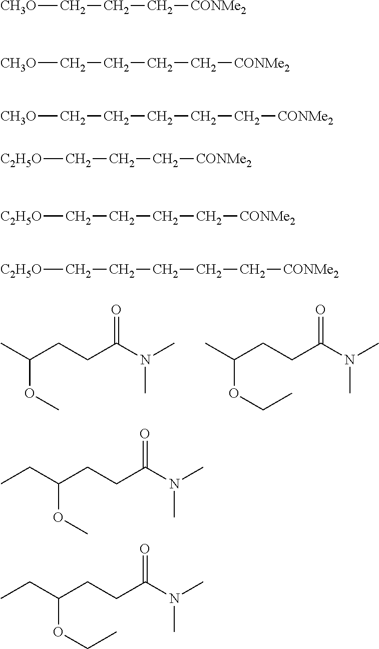

It is noted that the compound of the invention may be comprised in a material composition, comprising products other than the compound alone or in a mixture fitting formula (I). In the material composition, the compound of the invention may account for at least 10% by weight. Preferably, this is the main compound of the material composition. By main compound is meant in the present application, the compound for which the content is the highest, even if its content is less than 50% by weight (for example in a mixture of 40% of A, 30% of B, and 30% of C, the product A is the main compound). Even more preferably, the compound of the invention accounts for at least 50% by weight of the material composition, for example from 70% to 95% by weight, and even from 75% to 90% by weight. As indicated above, the material composition may be a reaction product. The other products of the material composition may notably be byproducts of impurities, un-reacted products, or products corresponding to reaction adducts of products comprised in the initial compounds not leading to the compounds of formula (I).

Method

The compound of the invention may be prepared by any suitable method.

These compounds may be synthesized via the following synthesis routes:
reaction between the lactones and the amines required for forming the corresponding amido-alcohols according to known operating procedures to one skilled in the art (see notably the references in Comprehensive Organic Transformations, 2^{nd} Edition, Richard C. Larock, page 1973-1976, or
reaction of etherification of the alcohol function of the amido-alcohol formed beforehand according to operating procedures known to one skilled in the art (see notably the references in Comprehensive Organic Transformations, 2^{nd} Edition, Richard C. Larock, page 890-898).

Active Phytosanitary Product a)

Active phytosanitary products, notably products non-soluble in water and solid are known to one skilled in the art. The active phytosanitary product may notably be a herbicide, an insecticide, an acaricide, a fungicide, a nematicide, a miticide, a molluscicide, an antimicrobial agent, or a rodenticide, for example a raticide.

It is noted that the phytosanitary formulation may comprise several different active phytosanitary products.

As non-limiting examples of suitable active materials, mention may be made inter alia of the products of the following groups:

Acetamide
Acidity regulator
Acylalanine
Aldehyde
Aliphatic ketone
Alkanamide
Alkane hydrocarbon
Alkyl iodide
Alkylamine
Alkylchlorophenoxy
Amide
Amidine
Amine
Anilide
Anilinopyrimidine
Anthranilic diamide
Aromatic carboxylic acid
Aromatic hydrocarbon
Aryaminopropionic acid
Aryl triazolinone
Arylalanine
Aryloxyalkanoic acid
Aryloxyphenoxypropionate
Aryloxyphenoxypropionic acid
Arylpyrrole
Auxin
Avermectin
Benonorbene
Benzamide
Benzanilide
Benzenamine
Benzene-dicarboxamide
Benzenedicarboxylic acid
Benzilate
Benzothiadiazole
Benzothiazole
Benzoylphenyl urea
Benzoylurea
Biopesticide
Biopesticide - nactins
Bipyridylium
Bis-carbamate
Botanical aldehyde
Bridged diphenyl
Carbamate
Carbamoyltriazole
Carbanilate
Carbonitrile
Carboxamide
Carboxylic acid
Cationic surfactant
Chlorinated acetal derivative of
glucose
Chlorinated aromatic
hydrocarbon
Chlorinated hydrocarbon
Chlorinated phenol
Chloroacetamide
Chloroacetanilide
Chloroaniline
Chloronitrile
Chlorophenoxy acid
Chlorophenoxy acid or ester
Chlorophenyl
Conazole
Coumarin
Coumarin anticoagulant
Cresol
Cyanoacetamide oxime
Cyanoimidazole
Cyclic dithiocarbamate
Cyclodiene
Cyclodiene, organochlorine
Cyclohexadione
Cyclohexadione oxime
Cyclohexanecarboxylate
derivative
Cyclohexanedione
Cyclohexanedione oxime
Cyclo-octane
Cyclopropanecarboxamide,
isomer mixture
Cytokinin
Diacylhydrazine
Dicarboximide
Dicarboxylicacid
Dimethyldithiocarbamate
Dimethylurea
Dinitroaniline
Dinitrophenol
Dinitrophenyl oxime
Diphenyl compound
Diphenyl ether
Diphenyl ether, sodium salt
Diphenyl oxazoline
Dithiocarbamate
Ethylene generator
Fenanilide
Formamidine
Furamide
Furanilide
Glycine derivative
Guanidine
Halogenated aliphatic
Halogenated aliphatic
compound
Halogenated alkane
Halogenated hydrocarbon
Heteroaramatic
Hydrazine carboxylate
Hydrocarbon
Hydrocoumarin
Hydroxyanilide
Hydroxybenzonitrile
Hydroxycoumarin
Imidazole
Imidazolinone
Indandione anticoagulant
Inert solvent
Inorganic compound
Inorganic metal oxide
Isomeric amine of pyridine
Isothiazolone
Isoxazole
Isoxazolidinone
Juvenile hormone analogue
Juvenile hormone mimic
Juvenile hormone mimic
(terpene)
Malonanilate
Mandelamide
Mercaptobenzothiazole
Methylthiotriazine
Methylthiotriazine
Mix of 3′O-ethyl-5,6-
dihydrospinosyn/3′-O-ethyl-
spinosyn
Mixture of alkylbenzyl
dimethylammonium chlorides
of various alkyl chain lengths
Monoterpene 1,4-cineole
Morphactin
Morpholine
Moulting hormone agonists
Neonicotinoid
Nereistoxin analogue
Nereistoxin analogue
insecticides
Nitrile
Nitroanisole
Nitrophenyl ether
Organoarsenic
Organochloride
Organochlorine
Organochlorine, racemic
mixture
Organometal
Organophosphate
Organophosphate racemic
mixture
Organophosphorus
Organothiophosphate
Organotin
Oxadiazine
Oxadiazolone
Oxadiazolone/phenylurea
Oxathiin
Oxazole
Oxazolidin
Oxidiazole
Oxime ether
Oxirane compound
Oxyacetamide
Petroleum derivative
Phenol
Phenoxipropionic acid
Phenyl urea
Phenylamide
Phenylfuranone
Phenylpyrazole
Phenylpyridazine
Phenylpyridinamine
Phenylpyrrole
Phenylurea
Pheromone
Phosphinic acid
Phosphonoglycine
Phosphonothioate
Phosphoramidate
Phosphorodithioate
Phosphorothiolate
Phosphorothiolate
Phthalamate
Phthalimide
Piperazine
Piperidine
Polycyclic aromatic ketone
Polysaccharide carbohydrate
Pryimidinamine
Pyrazole
Pyrazolone
Pyrethroid
Pyrethroid (diasterioisomer
mixture)
Pyrethroid ester
Pyrethroid, isomer mixture
Pyridazine
Pyridazinone
Pyridine
Pyridine carboxylic acid
Pyridine compound
Pyrimidine
Pyrimidine
Pyrimidinol
Pyrimidinyl carbinol
Pyrimidinyl carboxy compound
Pyrimidinyloxybenzoic
Pyrimidinyloxybenzoic
analogue
Pyrimidinylsulfonylurea
Quarternary ammonium
Quinazinalone
Quinazolinone
Quinoline
Quinolinecarboxylic acid
Quinolone
Quinone
Saturated fatty acid
Semicarbazone
Sodium nitrocompound
Sterol
Strobilurin
Strobilurin type-
methoxyacrylate
Substituted benzene
Sulfite ester
Sulfonamide
Sulfonanilide
Sulfonylurea
Sulfamide
Sulfamide
Surfactant
Synthetic pyrethroid
Tetracycline
Tetramic acid
Tetrazine
Tetrazolinone
Tetronic acid
Thiadiazolylurea
Thiazole
Thiazole carboximide
Thiazolidine
Thiocarbamate
Thiocarbamide
Thiol
Thiol compound
Thiophene
Thiourea
Triazine
Triazinone
Triazinylsulfonylurea
Triazole
Triazolepyrimidine
Triazolinone
Triazolinthione
Triazolobenzothiazole
Triazolone
Triazolopyrimidine
Triazolopyrimidine
sulfonamide
Triazopyrimidine sulfonamide
Triketone
Uracil
Uracil/amide
Urea

As non-limiting examples of suitable active materials, mention may be made inter alia of the following products:

1,2-benzisothiazolin-3-one
1,2-dibromoethane
1,2-dichloropropane
1,3-dichloropropene
1-dodecanol
1-methylcyclopropene
1-naphthylacetic acid
1O,1O′-
oxybisphenoxarsine
2-(thiocyanomethylthio)
benzothiazole
2,2-dibromo-3-
nitrilopropionamide
2,3,6-TBA
2,4,5-trichlorophenol
2,4,5-trichlorophenoxy-
acetique acid
2,4-D
2,4-DB
2,4-dimethylphenol
2,5-dichlorobenzoic acid
methyl ester
2,6-diisopropylnaphthalene
2-aminobutane
2-naphthyloxyacetic acid
2-phenylphenol
4-allylanisole
4-aminopyridine
4-CPA
5-methyl-2-(1-
methylethyl)phenol
6-benzylaminopurine
6-isopentenyl aminopurine
8-hydroxyquinoline sulfate
abamectin
acephate
acequinocyl
acetamiprid
acethion
acetic acid
acetochlor
acibenzolar-S-methyl
acifluorfen
acifluorfen-sodium
aclonifen
acrinathrin
acrolein
Adoxophyes orana
granulovirus
alachlor
alanycarb
albendazole
aldicarb
aldimorph
aldoxycarb
aldrin
allethrin
allidochlor
alloxydim
alpha-chlorohydrin
alpha-cypermethrin
alpha-naphthylthiourea
aluminium ammonium
sulfate
aluminium phosphide
aluminium sulfate
ametryn
amicarbazone
amidithion
amidosulfuron
aminocarb
aminopyralid
amisulbrom
amitraz
amitrole
ammonium acetate
ammonium carbonate
ammonium hydroxide
ammonium sulfamate
ammonium sulfate
ammonium thiocyanate
ampropylfos
ancymidol
anilazine
anilofos
anthracene oil
anthraquinone
aramite
asomate
asulam sodium
asulam
atrazine
aviglycine-HCl
azaconazole
azadirachtin
azafenidin
azamethiphos
azimsulfuron
azinphos-ethyl
azinphos-methyl
aziprotryne
azobenzene
azocyclotin
azoxybenzene
azoxystrobin
Bacillus sphaericus
Bacillus subtilis
Bacillus thuringiensis
barban
barium polysulfide
Beauveria bassiana
beflubutamid
benalaxyl
benalaxyl-M
benazolin ethyl
benazolin
bendiocarb
benfluralin
benfuracarb
benfuresate
benodanil
benomyl
benoxacor
benquinox
bensulfuron
bensulfuron-methyl
bensulide
bensultap
bentaluron
bentazone
benthiavalicarb
benzalkonium chloride
benzfendizone
benzobicyclon
benzofenap
benzoic acid
benzoximate
benzoylprop
benzoylprop-ethyl
benzthiazuron
benzyl benzoate
beta-cyfluthrin
beta-cypermethrin
bifenazate
bifenox
bifenthrin
bilanafos
bilanafos-sodium
binapacryl
bioallethrin
bioresmethrin
biphenyl
bispyribac-sodium
bis-trichloromethyl sulfone
bistrifluron
bitertanol
bixafen
blasticidin-S
bone oil
Bordeaux mixture
boscalid
brodifacoum
bromacil
bromadiolone
bromethalin
bromobutide
bromocyclen
bromofenoxim
bromomethane
bromophos
bromophos-ethyl
bromopropylate
bromoxynil heptanoate
bromoxynil octanoate
bromoxynil
bromuconazole
bronopol
bufencarb
buminafos
bupirimate
buprofezin
butacarb
butachlor
butafenacil
butanethiol
butocarboxim
butoxycarboxim
butralin
butroxydim
buturon
butylate
cadusafos
cafenstrole
calciferol
calcium carbide
calcium carbonate
calcium chloride
calcium hydroxide
calcium phosphate
calcium phosphide
camphechlor
capsaicin
captafol
captan
carbaryl
carbendazim
carbetamide
carbofuran
carbon dioxide
carbon tetrachloride
carbophenothion
carbosulfan
carboxin sulfoxide
carboxin
carfentrazone-ethyl
carpropamid
cartap
chinomethionat
chitosan
chlomethoxyfen
chloralose
chloramben
chloranil
chlorantraniliprole
chlorbenside
chlorbromuron
chlorbufam
chlordane
chlordecone
chlordimeform
chloretazate
chlorethoxyfos
chlorfenac
chlorfenapyr
chlorfenethol
chlorfenprop-methyl
chlorfenson
chlorfensulfide
chlorfenvinphos
chlorfluazuron
chlorflurenol
chloridazon
chlorimuron-ethyl
chlormephos
chlormequat chloride
chlorobenzilate
chloromethiuron
chloroneb
chlorophacinone
chlorophyllin
chloropicrin
chloropropylate
chlorothalonil
chlorotoluron
chloroxuron
chloroxylenol
chlorphonium chloride
chlorphoxim
chlorpropham
chlorpyrifos
chlorpyrifos-methyl
chlorsulfuron
chlorthal-dimethyl
chlorthiamid
chlorthion
chlorthiophos
chlozolinate
cholecalciferol
choline chloride
chromafenozide
cinidon-ethyl
cinmethylin
cinnamaldehyde
cinosulfuron
citric acide anhydrous
citronella huile
clethodim
clodinafop
clodinafop-propargyl
cloethocarb
clofencet
clofentezine
clomazone
cloprop
clopyralid
cloquintocet-mexyl
cloransulam-methyl
clothianidin
codlemone
coniothyrium minitans
copper (1) oxide
copper II acetate
copper II carbonate
copper II chloride
copper II hydroxide
copper oxychloride
copper sulfate
coumachlor
coumafuryl
coumaphos
coumatetralyl
crimidine
crotoxyphos
crufomate
cufraneb
cuprobam
cyanamide
cyanazine
cyanofenphos
cyanophos
cyazofamid
cyclanilide
cycloate
cycloprothrin
cyclosulfamuron
cycloxydim
cycluron
Cydia pomonella
granulosis virus
cyflufenamid
cyflumetofen
cyfluthrin
cyhalofop
cyhalofop-butyl
cyhalothrin
cyhexatin
cymiazol
cymoxanil
cypermethrin
cyphenothrin
cyproconazole
cyprodinil
cyprofuram
cyprosulfamide
cyromazine
dalapon
dalapon-sodium
daminozide
dazomet
DDT
deltamethrin
demeton
demeton-O-methyl sulfone
demeton-O-methyl
demeton-S-methyl sulfone
demeton-S-methyl
denatonium benzoate
desmedipham
desmetryn
diafenthiuron
dialifos
di-allate
diazinon
dibromochloropropane
dicamba
dichlobenil
dichlofenthion
dichlofluanid
dichlone
dichlormid
dichlorophen
dichlorprop
dichlorprop-P
dichlorvos
diclobutrazol
diclofop
diclofop-methyl
diclomezine
dicloran
diclosulam
dicofol
dicrotophos
dicyclanil
dicyclopentadiene
didecyldimethylammonium
chloride
dieldrin
dienochlor
diethofencarb
diethyltoluamide
difenacoum
difenoconazole
difenoxuron
difenzoquat
difethialone
diflovidazin
diflubenzuron
diflufenican
diflufenzopyr
diflumetorim
difunon
dikegulac
dikegulac-sodium
dimefox
dimefuron
dimepiperate
dimethachlor
dimethametryn
dimethenamid
dimethenamid-P
dimethipin
dimethirimol
dimethoate
dimethomorph
dimethrin
dimethylvinphos
dimexano
dimoxystrobin
diniconazole
dinitramine
dinobuton
dinocap
dinoseb
dinotefuran
dinoterb
dioxabenzophos
dioxacarb
dioxathion
diphacinone
diphenamid
diphenylamine
diquat dibromide
diquat
disulfoton
ditalimfos
dithianon
dithiopyr
diuron
DNOC
dodecyl acetate
dodemorph acetate
dodemorph
dodine
edifenphos
emamectin benzoate
empenthrin
endosulfan
endothal
endothion
endrin
EPN
epoxiconazole
eprinomectin
EPTC
esfenvalerate
esprocarb
etaconazole
ethaboxam
ethalfluralin
ethametsulfuron-methyl
ethanedial
ethanethiol
ethephon
ethidimuron
ethiofencarb
ethion
ethiprole
ethirimol
ethoate-methyl
ethofumesate
ethoprophos
ethoxyquin
ethoxysulfuron
ethylene bisisothiocyanate
sulfide
etofenprox
etoxazole
etridiazole
etrimfos
famoxadone
famphur
farmesene
fatty acides
fenamidone
fenaminosulf
fenamiphos
fenarimol
fenazaflor
fenazaquin
fenbuconazole
fenbutatin oxide
fenchlorazole
fenchlorazole-ethyl
fenchlorphos
fenclorim
fenfuram
fenhexamid
fenitrothion
fenobucarb
fenoprop
fenothiocarb
fenoxanil
fenoxaprop-ethyl
fenoxaprop-P-ethyl
fenoxycarb
fenpiclonil
fenpropathrin
fenpropidin
fenpropimorph
fenpyroximate
fenson
fensulfothion
fenthion
fentin acetate
fentin chloride
fentin hydroxide
fentrazamide
fenuron
fenvalerate
ferbam
ferric phosphate
fipronil
flamprop
flamprop-M-isopropyl
flazasulfuron
flocoumafen
flonicamid
florasulam
fluacrypyrim
fluazifop-butyl
fluazifop-P-butyl
fluazinam
fluazolate
fluazuron
flubendiamide
flubenzimine
flucarbazone-sodium
flucetosulfuron
fluchloralin
flucycloxuron
flucythrinate
fludioxonil
flufenacet
flufenoxuron
flufenpyr-ethyl
flumequine
flumethrin
flumetralin
flumetsulam
flumiclorac-pentyl
flumioxazine
flumorph
fluometuron
fluopicolide
fluopyram
fluoroacetamide
fluorodifen
fluoroglycofen
fluotrimazole
fluoxastrobin
flupoxam
flupropanate-sodium
flupyrsulfuron
flupyrsulfuron-methyl
fluquinconazole
flurazole
flurenol
fluridone
flurochloridone
fluroxypyr
fluroxypyr-meptyl
flurprimidol
flurtamone
flusilazole
flusulfamide
fluthiacet methyl
flutolanil
flutriafol
fluxofenim
folpet
fomesafen
fonofos
foramsulfuron
forchlorfenuron
formaldehyde
formetanate
formothion
fosamine
fosetyl
fosetyl-aluminium
fospirate
fosthiazate
fosthietan
fuberidazole
furalaxyl
furametpyr
furathiocarb
furfural
furilazole
furmecyclox
gamma-cyhalothrin
gibberellic acide
glufosinate
glufosinate-ammonium
glutaraldehyde
glyphosate trimesium
glyphosate
gossyplure
guazatine
halfenprox
halofenozide
halosulfuron-methyl
haloxyfop
haloxyfop-etotyl
haloxyfop-P
haloxyfop-P-methyl
heptachlor
heptenophos
hexachlorobenzene
hexachlorophene
hexaconazole
hexadecanoic acide
hexaflumuron
hexazinone
hexythiazox
hydramethylnon
hydrogen peroxide
hydroprene
hymexazol
icaridin
imazalil
imazamethabenz
imazamethabenz-methyl
imazamox
imazapic
imazapyr
imazaquin
imazethapyr
imazosulfuron
imibenconazole
imicyafos
imidacloprid
iminoctadine triacetate
iminoctadine
imiprothrin
indanofan
indolylacetic acide
indolylbutyric acide
indoxacarb
iodofenphos
iodomethane
iodosulfuron
iodosulfuron-methyl-
sodium
ioxynil octanoate
ioxynil
ipconazole
iprobenfos
iprodione
iprovalicarb
iron sulfate
isazofos
isobenzan
isocarbophos
isodrin
isofenphos-methyl
isopolinate
isoprocarb
isopropalin
isoprothiolane
isoproturon
isopyrazam
isotianil
isoval
isoxaben
isoxadifen ethyl
isoxaflutole
isoxathion
ivermectin
jasmone
kaolin
karbutilate
kasugamycin hydrochloride
hydrate
kelevan
kinetin
kinoprene
kresoxim-methyl
lactofen
lambda-cyhalothrin
laminarin
L-carvone
lenacil
leptophos
limonene
lindane
linuron
lithium perfluorooctane
sulfonate
lufenuron
magnesium phosphide
malathion
maleic hydrazide
mancopper
mancozeb
mandipropamid
maneb
MCPA
MCPA-thioethyl
MCPB
mecarbam
mecoprop
mecoprop-P
mefenacet
mefenpyr
mefluidide
menadione
menazon
mepanipyrim
mephosfolan
mepiquat chloride
mepiquat
mepronil
meptyldinocap
merphos
mesosulfuron
mesosulfuron-methyl
mesotrione
metaflumizone
metalaxyl
metalaxyl-M
metaldehyde
metamifop
metamitron
metam-sodium
Metarhizium anisopliae
metazachlor
metconazole
methabenzthiazuron
methacrifos
methamidophos
methasulfocarb
methazole
methfuroxam
methidathion
methiocarb
methomyl
methoprene
methoprotryne
methoxychlor
methoxyfenozide
methyl anthranilate
methyl nonyl ketone
methylarsonic acide
metiram
metobromuron
metofluthrin
metolachlor
metominostrobin
metosulam
metoxuron
metrafenone
metribuzin
metsulfovax
metsulfuron
metsulfuron-methyl
mevinphos
mexacarbate
milbemectin
mirex
molinate
monalide
monocrotophos
monolinuron
monosodium
methylarsonate
monuron
morphothion
muscalure
myclobutanil
nabam
naled
naphthalene
naproanilide
napropamide
naptalam
neburon
nepetalactone
nicosulfuron
nicotine
nitenpyram
nitralin
nitrapyrin
nitrofen
nitrothal isopropyl
N-methylneodecanamide
norbormide
norflurazon
noruron
novaluron
noviflumuron
nuarimol
nuranone
ocimene
octanal
octhilinone
ofurace
olein
omethoate
orbencarb
orthosulfamuron
orysastrobin
oryzalin
oxabetrinil
oxadiargyl
oxadiazon
oxadixyl
oxamyl
oxasulfuron
oxaziclomefone
oxpoconazole fumarate
oxycarboxin
oxydemeton-methyl
oxyfluorfen
oxytetracycline
paclobutrazol
Paecilomyces
fumosoroseus
Paecilomyces lilacinus
paraffin oil (C11-C25) [4a]
paraffin oil (C11-C30) [4c]
paraffin oil (C15-C30) [4b]
paraffin oil (C17-C31) [2]
paraffin oil (C18-C30) [1,
not ASU]
paraffin oil [3 Not NEU]
paraquat dichloride
paraquat
parathion
parathion-methyl
pebulate
pelargonic acide
penconazole
pencycuron
pendimethalin
penoxsulam
pentachlorophenol
pentacosane
pentanochlor
penthiopyrad
pentoxazone
permethrin
peroxyacetic acide
pethoxamid
phenkapton
phenmedipham
phenothrin
phenthoate
Phlebiopsis gigantea
phorate
phosacetim
phosalone
phosmet
phosnichlor
phosphamidon
phosphine
phoxim
phthalide
picloram
picolinafen
picoxystrobin
pinoxaden
piperalin
piperidine
piperonyl butoxide
piperophos
pirimicarb
pirimiphos-ethyl
pirimiphos-methyl
p-menthane-3,8-diol
polynactins
polyoxin
potassium bicarbonate
potassium iodide
potassium thiocyanate
prallethrin
precocene II
pretilachlor
primisulfuron methyl
primisulfuron
prochloraz
procymidone
prodiamine
profenofos
profoxydim
prohexadione-calcium
promecarb
prometon
prometryn
propachlor
propamocarb hydrochloride
propamocarb
propanil
propaquizafop
propargite
propazine
propetamphos
propham
propiconazole
propineb
propionic acid
propisochlor
propoxur
propoxycarbazone
propoxycarbazone-sodium
propyzamide
proquinazid
prosulfocarb
prosulfuron
prothiocarb
prothioconazole
prothiofos
prothoate
PT807-HCI
putrescine
pymetrozine
pyraclofos
pyraclonil
pyraclostrobin
pyraflufen
pyraflufen-ethyl
pyrasulfotole
pyrazophos
pyrazosulfuron-ethyl
pyrazoxyfen
pyrethrins
pyribenzoxim
pyridaben
pyridafenthion
pyridafol
pyridalyl
pyridate
pyrifenox
pyrifluquinazon
pyriftalid
pyrimethanil
pyriminobac-methyl
pyrimisulfan
pyriprole
pyriproxyfen
pyrithiobac-sodium
pyroquilone
pyroxasulfone
pyroxsulam
quartz sand
quinalphos
quinclorac
quinmerac
quinoclamine
quinoxyfen
quintozene
quizalofop-ethyl
quizalofop-P-ethyl
quizalofop-P-tefuryl
resmethrin
rimsulfuron
rotenone
sabadilla
saflufenacil
scilliroside
sebuthylazine
secbumeton
sethoxydim
siduron
silica
silthiofam
simazine
simeconazole
simetryn
sintofen
S-methoprene
S-metolachlor
sodium 5-nitroguaiacolate
sodium carbonate
sodium chlorate
sodium chloride
sodium hypochlorite
sodium o-nitrophenolate
sodium p-nitrophenolate
spinetoram
spinosad
spirodiclofen
spiromesifen
spirotetramat
spiroxamine
streptomycin
strobane
sucrose octanoate
sulcotrione
sulfentrazone
sulfluramid
sulfometuron-methyl
sulfosulfuron
sulfotep
sulfuryl fluoride
sulfur
sulfuric acid
sulprofos
tau-fluvalinate
TCA-sodium
TDE
tebuconazole
tebufenozide
tebufenpyrad
tebupirimfos
tebutam
tebuthiuron
tecloftalam
tecnazene
teflubenzuron
tefluthrin
tembotrione
temephos
tepraloxydim
terbacil
terbufos
terbumeton
terbuthylazine
terbutryn
tetrachlorvinphos
tetraconazole
tetradifon
tetraethyl pyrophosphate
tetramethrin
tetrasul
thenylchlor
thiabendazole
thiacloprid
thiamethoxam
thiazafluron
thiazopyr
thidiazuron
thiencarbazone-methyl
thifensulfuron
thifensulfuron-methyl
thifluzamide
thiobencarb
thiocyclam hydrogen
oxalate
thiocyclam
thiodicarb
thiofanox
thiometon
thionazin
thiophanate-methyl
thiosultap
thiosultap-disodium
thiosultap-monosodium
thiourea
thiram
tiocarbazil
tolclofos-methyl
tolylfluanid
topramezone
tralkoxydim
tralomethrin
transfluthrin
triadimefon
triadimenol
tri-allate
triapenthenol
triasulfuron
triazamate
triazophos
triazoxide
tribenuron
tribenuron-methyl
tribufos
trichlorfon
trichloronate
triclopyr
tricosane
tricyclazole
tridemorph
tridiphane
trietazine
trifloxystrobin
trifloxysulfuron
trifloxysulfuron-sodium
triflumizole
triflumuron
trifluralin
triflusulfuron
triflusulfuron-methyl
triforine
trimedlure
trimethacarb
trinexapac-ethyl
triticonazole
tritosulfuron
uniconazole
urea sulfate
validamycin
vamidothion
vernolate
verticillium lecanii
vinclozolin
warfarin
XMC
zeatin
zeta-cypermethrin
zinc oxide
zineb
ziram
zoxamide

From this list, are preferably selected non-water-soluble products.

These products and names are known to one skilled in the art. Several active phytosanitary products may be associated together.

Optional Surfactant (c)

The phytosanitary formulation may comprise at least one surfactant. It may comprise a mixture of several different surfactants. The surfactant may facilitate emulsification or dispersion after putting the formulation in presence with water, and/or to stabilize (over time and/or in temperature) the formulation or the dispersion, for example by avoiding sedimentation.

The surfactants are known compounds, which have a molar mass which is generally smaller, for example less than 1,000 g/mol. The surfactant may be an anionic surfactant in a salified or acid form, a non-ionic, preferably polyalcoxylated surfactant, a cationic or an amphoteric surfactant (term also including zwitterionic surfactants). This may be a mixture or a combination of these surfactants.

As examples of anionic surfactants, mention may be made, without any intention of being limited thereto:
alkylsulfonic acids, arylsulfonic acids, optionally substituted with one or several hydrocarbon groups, and the acid function of which is partly or totally salified, like C₈-C₅₀, more particularly C₈-C₃₀, preferably C₁₀-C₂₂ alkylsulfonic acids, benzenesulfonic acids, naphthalenesulfonic acids, substituted with one to three C₁-C₃₀, preferably C₄-C₁₆ aklyl, and/or C₂-C₃₀, preferably C₄-C₁₆ alkenyl groups.
mono- or di-esters of alkylsulfosuccinic acids, the linear or branched alkyl portion of which, optionally substituted with one or more linear or branched C₂-C₄ hydroxylated and/or alkoxylated (preferably ethoxylated, propoxylated, ethopropoxylated).
ester phosphates more particularly selected from those comprising at least one saturated, unsaturated or aromatic, linear or branched hydrocarbon group comprising from 8 to 40 carbon atoms, preferably 10 to 30, optionally substituted with at least one alkoxylated (ethoxylated, propoxylated, ethopropoxylated) group. Further, they comprise at least one mono- or di-esterified ester phosphate group so that it is possible to have one or two free acid or partly or totally salified groups. Preferred ester phosphates are of the type of mono- and di-esters of phosphoric acid and of alkoxylated (ethoxylated and/or propoxylated) mono-, di- or tri-styrylphenol, or of alkoxylated (ethoxylated and/or propoxylated), mono-, di- or tri-alkylphenol optionally substituted with one or four alkyl groups; of phosphoric acid and of a C₈-C₃₀ alcohol, preferably alkoxylated (ethoxylated or ethopropoxylated) C₁₀-C₂₂ alcohol; of phosphoric acid and of a C₈-C₂₂ alcohol, preferably non-alkoxylated C₁₀-C₂₂ alcohol.
the ester sulfates obtained from saturated, or aromatic alcohols optionally substituted with one or several alkoxylated (ethoxylated, propoxylated, ethopropoxylated) groups, and for which the sulfate functions appear in free acid form, or partly or totally neutralized. As an example, mention may be made of ester sulfates obtained more particularly from saturated or unsaturated C₈-C₂₀ alcohols, which may comprise 1 to 8 alkoxylated (ethoxylated, propoxylated, ethopropoxylated) units; ester sulfates obtained from polyalcoxylated phenol, substituted with 1 to 3 saturated or unsaturated C₂-C₃₀ hydroxycarbon groups, and wherein the number of alkoxylated units is comprised between 2 and 40; the ester sulfates obtained from polyalcoxylated mono-, di- or tri-styrylphenol wherein the number of alkoxylated units varies from 2 to 40.

Anionic surfactants may be in an acid form (they are potentially anionic), or in a partly or totally salified form with a counter-ion. The counter-ion may be an alkaline metal, such as sodium or potassium, an earth-alkaline metal, such as calcium, or an ammonium ion of formula N(R)₄⁺ wherein R, identical or different, represent a hydrogen atom or a C₁-C₄ alkyl radical optionally substituted with an oxygen atom.

As examples of non-ionic surfactants, mention may be made, without intending to be limited thereto:
polyalcoxylated (ethoxylated, propoxylated, ethopropoxylated) phenols substituted with at least one C₄-C₂₀, preferably C₄-C₁₂ alkyl radical or substituted with at least one alkylaryl radical, the alkyl portion of which is a C₁-C₆ alkyl. More particularly, the total number of alkoxylated units is comprised between 2 and 100. As an example, mention may be made of polyalcoxylated mono-, di- or tri-(phenylethyl) phenols, or polyalcoxylated nonylphenols. From among the ethoxylated and/or propoxylated, sulfated and/or phosphated di- or tri-styrylphenols, mention may be made of ethoxylated di-(phenyl-1-ethyl)phenol, containing 10 oxyethylene units, ethoxylated di-(phenyl-1-ethyl)phenol, containing 7 oxyethylene units, sulfated ethoxylated di-(phenyl-1-ethyl)phenol, containing 7 oxyethylene units, ethoxylated tri-(phenyl-1-ethyl)phenol, containing 8 oxyethylene units, ethoxylated tri-(phenyl-1-ethyl)phenol, containing 16 oxyethylene units, sulfated ethoxylated tri-(phenyl-1-ethyl)phenol, containing 16 oxyethylene units, ethoxylated tri-(phenyl-1-ethyl)phenol, containing 20 oxyethylene units, phosphated ethoxylated tri-(phenyl-1-ethyl)phenol, containing 16 oxyethylene units.
polyalkoxylated (ethoxylated, propoxylated, ethopropoxylated) C₆-C₂₂ alcohols or fatty acids. The number of alkoxylated units is comprised between 1 and 60. The term of ethoxylated fatty acid includes both products obtained by ethoxylation of a fatty acid with ethylene oxide and those obtained by esterification of a fatty acid by a polyethylene glycol.
polyalkoxylated (ethoxylated, propoxylated, ethopropoxylated)triglycerides of plant or animal origin. Thus suitable triglycerides from lard, tallow, groundnut oil, butter oil, cottonseed oil, flax oil, olive oil, palm oil, grape pip oil, fish oil, soya bean oil, castor oil, rapeseed oil, copra oil, coconut oil, and comprising a total number of alkoxylated units comprised between 1 and 60. The term of ethoxylated triglyceride is both directed to the products obtained by ethoxylation of a triglyceride with ethylene oxide and those obtained by trans-esterification of a triglyceride with a polyethylene glycol.
optionally polyalkoxylated (ethoxylated, propoxylated, ethopropoxylated) sorbitan esters, more particularly cyclized sorbitol esters of C₁₀-C₂₀ fatty acids like lauric acid, stearic acid or oleic acid, and comprising a total number of alkoxylated units comprised between 2 and 50.

Useful emulsifiers are notably the following products, all marketed by Rhodia:
Soprophor® TSP/724: surfactant based on ethopropoxylated tristyrylphenol
Soprophor® 796/0: surfactant based on ethopropoxylated tristyrylphenol
Soprophor® CY 8: surfactant based on ethoxylated tristyrylphenol
Soprophor® BSU: surfactant based on ethoxylated tristyrylphenol
Alkamuls® RC: tensioactif based on ethoxylated castor oil
Alkamuls® OR/36: tensioactif based on ethoxylated castor oil
Alkamuls® T/20: tensioactif based on sorbitan ester
Geronol® TBE-724: mixture of surfactants comprising an ethopropoxylated non-ionic surfactant.

When it contains some of them, the formulation of the invention comprises advantageously at least 4%, preferably at least 6%, preferably at least 10%, and preferentially at least 15%, by weight of dry material, at least one surfactant c).

### Other Details as Regards the Phytosanitary Formulation

The concentrated phytosanitary formulation preferably does not comprise significant amounts of water. Typically the water content is less than 50% by weight, advantageously less than 25% by weight, it may be generally less than 10% by weight.

The formulation is preferably a liquid formulation, for example in the form of an emulsifiable concentrate (EC), of a liquid concentrate (SL), of a concentrated emulsion (EW), of a suspo-emulsion (SE), or a micoremulsion (ME).

In this case, it preferably comprises at least 500 g/L of water, plus preferably less than 250 g/L. It will generally be less than 100 g/L.

The formulations may advantageously comprise:

a) from 5 to 70%, preferably from 10 to 50%, of the phytosanitary product by weight of active material,

b) from 10 to 95%, preferably from 20 to 80%, of the solvent by weight,

c) from 0 to 60%, preferably from 5 to 50%, preferably from 8 to 25%, by weight of dry material, of surfactant,

d) from 0 to 10% by weight of water.

The making of solid formulations is not excluded, for example formulations in which a liquid comprising the phytosanitary product solubilised in the solvent, is supported by a mineral and/or dispersed in a solid matrix.

The formulation may of course comprise other ingredients (or “other additives”) than the active phytosanitary product, a compound of formula (I), the optional surfactant(s) and the optional water. It may notably be comprise co-solvents, dispersant agents, agents for modifying viscosity, flow property control agents, fertilizers, antifoam agents, notably silicone antifoam agents, anti-bounce agents, anti-leaching agents, inert fillers, notably mineral fillers, antifreeze agents, crystallisation inhibitors such as non-polyalkoxylated fatty acids or fatty alcohols, for example the product Alkamuls® OL700 marketed by Rhodia, mixtures or associations thereof.

### Co-Solvent

According to a particular embodiment, the compound of the invention is used in the phytosanitary formulation as a co-solvent, in combination with another solvent or a mixture of other solvents. The other co-solvent(s) may subsequently be designated as co-solvent(s). The ratio by weight between the compound of the invention and the other solvent may notably be comprised between 10/90 and 90/10, for example between 10/50 and 50/50 or between 50/50 and 90/10.
As examples of other solvents, which may be applied, mention is notably made of:

aliphatic solvents,

paraffins with branched or linear chains

cyclic hydrocarbons

aromatic solvents

phosphorus-containing solvents

sulfur-containing solvents

nitrogen-containing solvents

aliphatic mono-, di- or tri-esters

cyclic esters

cyclic, aliphatic and/or aromatic ketones

alkylcyclohexanones

dialkylketones

acetoacetates

benzylketones

acetophenone

alcohols

cycloalcohols

glycols, glycol ethers, and their polymers

propylene glycols

glycol ether acetates

aromatic alcohols

carbonates

ethers

halogenated solvents.

Our most particularly preferred:
alkyl benzene and alkyl naphthalenes,
the product Solvesso® 100, 150, 200 in their standard versions and ND versions
alkanolamides and their alkyl ethers,
fatty acids and their alkyl esters, notably methyl esters, for example methyl oleate,
alkyldimethylamides
N-alkyl-pyrrolidones, notably N-methyl-pyrrolidone and N-ethyl-pyrrolidone
trialkylphosphates
linear or branched aliphatic alcohols and their esters
di-esters of dicarboxylic acids
linear or branched paraffins
white oils
glycols and glycol ethers
acetophenone
butyrolactone
DMSO.

### Preparation and Use of the Formulation

Conventional methods for preparing phytosanitary formulations or mixtures of solvents may be applied. It is possible to proceed by simple mixing of the constituents.
The concentrated phytosanitary formulation is intended to be spread over a cultivated field or one to be cultivated, for example a soya bean field, most often after dilution in water, in order to obtain a diluted composition. The dilution is generally performed by the farmer, directly in a tank (“tank-mix”), for example in the tank of a device intended to spread the composition. It is not excluded that the farmer adds other phytosanitary products, for example fungicides, herbicides, pesticides, insecticides, fertilizers. Thus, the formulation may be used for preparing a composition diluted in water of the active phytosanitary product, by mixing at least one part by weight of concentrated formulation with at least 10 parts of water, preferably less than 1,000 parts. The dilution levels and the amount to be applied on the field generally depend on the phytosanitary product and on the desirable dose for treating the field; this may be determined by the farmer.
Other details or advantages may become apparent upon considering the examples which follow, without any limitation.

### EXAMPLES

### Example 1.1

### Preparation of a hydroxydimethylamide of formula HO—(CH₂)ₙ—C(═O)—N(CH₃)₂ (Compound A)

The synthesis scheme is the following:

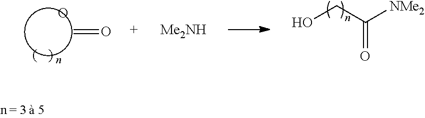

The typical operating procedure, for n ranging from 3 to 5, is the following.

Into a 500 ml flask containing the lactone (1 mole) is poured within about 1 hour and at +25° C. dimethylamine in an aqueous solution (60% by mass) (1.5 moles). The mixture is maintained with stirring at this temperature until complete consumption of the lactone (about 3 to 4 hours). The reaction crude is then neutralized by adding a 37% by mass hydrochloric acid aqueous solution until a pH of 7 is obtained. The aqueous phase is then extracted (3*100 ml) with an organic solvent non-miscible with water (dichloromethane or ethyl acetate). The organic phases are collected, dried on sodium sulfate and the solvent is evaporated in a partial vacuum. The residue is engaged as such in the next functionalization step.

### Example 1.2

### Preparation of a methoxyalkyldimethylamide of formula CH₃O—(CH₂)ₙ—C(═O)—N(CH₃)₂

The synthesis scheme is the following:

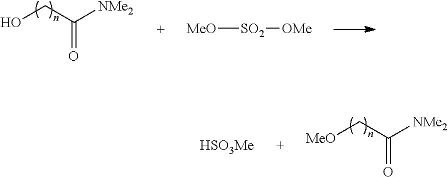

The operating procedure for n=3 is the following.

In a 2,000 ml flask containing the hydroxyamide (compound A3) (435 g; 3.28 moles) and THF (960 g) is added powdered soda (170 g; 4.22 moles) at +25° C. On this mixture, dimethylsulfate (508 g; 3.99 moles) is poured within 1.5 hours by maintaining the reaction medium temperature below +45° C. The mixture is maintained at +50° C. with stirring until complete consumption of the initial hydroxyamide (about 20 hours). The reaction crude is then diluted with water (1633 g) and dichloromethane (1325 g). The organic phase is separated from the aqueous phase. The latter is extracted twice with about 1300 g of dichloromethane. The organic phases are collected, dried on sodium sulfate and the solvent is evaporated in a partial vacuum. The desired product (425 g) is then obtained with a purity greater than 98% (yield: 89%).

The operating procedure for n=4 is the following.

Into a 1,000 ml flask containing the hydroxyamide (compound A4) (246 g; 1.7 moles) and THF (500 g) is added powdered soda (87.2 g; 2.18 moles) at +25° C. On this mixture, dimethylsulfate (265 g; 2.07 moles) is poured within 1.5 hours while maintaining the temperature of the reaction medium below +45° C. The mixture is maintained at +50° C. with stirring until complete consumption of the initial hydroxyamide (about 20 hours). The reaction crude is then diluted with water (1200 g) and dichloromethane (1200 g). The organic phase is separated from the aqueous phase. The latter is extracted twice with about 1200 g of dichloromethane. The organic phases are collected, dried on sodium sulfate and the solvent is evaporated in a partial vacuum. The desired product (216 g) is then obtained with a purity of the order of 98% (yield: 80%).

The operating procedure for n=5 is the following.

Into a 2,000 ml flask containing the hydroxyamide (compound A5) (334 g; 2.1 moles) and THF (600 g) is added the powered soda (108 g; 2.7 moles) at +25° C. On this mixture, dimethylsulfate (327 g; 2.56 moles) is poured within 1.5 hours while maintaining the temperature of the reaction medium below +45° C. The mixture is maintained at +50° C. with stirring until complete consumption of the initial hydroxyamide (about 20 hours). The reaction crude is then diluted with water (1300 g) and dichloromethane (1300 g). The organic phase is separated from the aqueous phase. The latter is extracted twice with about 1200 g of dichloromethane. The organic phases are collected, dried on sodium sulfate and the solvent is evaporated in a partial vacuum. The desired product (330 g) is then obtained with purity greater than 98% (yield: 91%).

The table below summarizes the different synthesized products, the yields and their purities.

| Compound | N | Yield (%) | Purity (%) |
|---|---|---|---|
| 1 | 3 | 89 | <98 |
| 2 | 4 | 80 | 98 |
| 3 | 5 | 91 | <98 |

### Example 1.3

### Preparation of an ethoxyalkyldimethylamide of formula CH₃—CH₂O—(CH₂)ₙ—C(═O)—N(CH₃)₂

The synthesis scheme is the following:

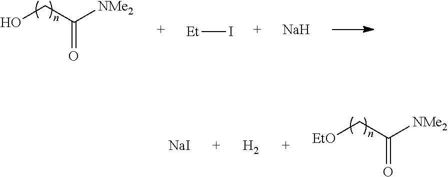

The operating procedure for n=3 is the following.

Into a 250 ml flask containing de-oiled sodium hydride (1.45 g; 60.5 mmol) in THF (130 g) maintained at 0° C., the preceding hydroxyamide A3 (5.89 g; 45.0 mmol) is added within 30 minutes. To this mixture, iodoethane (10.4 g; 66.4 mmol) is added within 30 minutes and at 0° C. The mixture is maintained at +25° C. with stirring until complete consumption of the initial hydroxyamide (about 20 hours). The reaction crude is then diluted with water (50 g) and dichloromethane (150 g). The organic phase is successively washed with a saturated ammonium chloride solution (50 ml), sodium hydrogencarbonate (50 ml) and water (50 ml). The organic phase is dried on sodium sulfate and the solvent is evaporated in a partial vacuum. The desired product (4.0 g) is then obtained with a purity of the order of 97.5% (yield: 56%).

The operating procedure for n=4 is the following.

Into a 250 ml flask containing de-oiled sodium hydride (1.14 g; 47.5 mmol) in THF (172 g) maintained at 0° C., the preceding hydroxyamide A4 (5.23 g; 35.3 mmol) is added within 30 minutes. To this mixture, iodoethane (7.46 g; 52.1 mmol) is added within 30 minutes and at 0° C. The mixture is maintained at +25° C. with stirring until complete consumption of the initial hydroxyamide (about 20 hours). The reaction crude is then diluted with water (50 g) and with dichloromethane (150 g). The organic phase is successively washed with an ammonium chloride saturated solution (50 ml), with sodium hydrogencarbonate (50 ml) and water (50 ml). The organic phase is dried on sodium sulfate and the solvent is evaporated in partial vacuum. The desired product (2.8 g) is then obtained with a purity greater than 98% (yield: 48%).

The operating procedure for n=5 is the following.

Into a 250 ml flask containing de-oiled sodium hydride (1.03 g; 43.2 mmol) in THF (150 g) maintained at 0° C., the preceding hydroxyamide A5 (5.1 g; 32.1 mmol) is added within 30 minutes. To this mixture, iodoethane (7.4 g; 47.4 mmol) is added within 30 minutes and at 0° C. The mixture is maintained at +25° C. with stirring until complete consumption of the initial hydroxyamide (about 20 hours). The reaction crude is then diluted with water (50 g) and dichloromethane (150 g). The organic phase is successively washed with a saturated ammonium chloride solution (50 ml), sodium hydrogencarbonate (50 ml) and water (50 ml). The organic phase is dried on sodium sulfate and the solvent is evaporated in partial vacuum. The desired product (1.9 g) is then obtained with a purity greater than 98% (yield: 32%).

The table below summarizes the different synthesized products, the yields and their purities.

| Compound | n | Yield (%) | Purity (%) |
|---|---|---|---|
| 4 | 3 | 56 | 97.5 |
| 5 | 4 | 48 | <98 |
| 6 | 5 | 32 | <98 |

The same synthesis procedures were carried out for preparing branched derivatives by using as a raw material the corresponding lactones.

| Compound | Structure |
|---|---|
| 7 | 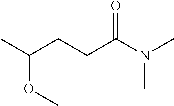 |
| 8 | 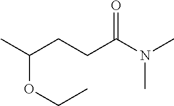 |
| 9 | 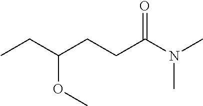 |
| 10 | 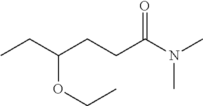 |

### Example 2

### Phytosanitary Formulations

By mixing the ingredients, formulations of various phytosanitary active ingredients of the type emulsifiable concentrate type (EC) are prepared.
The formulations comprise:

the active ingredient, in an amount by weight (of active material) indicated in the table below,

10% by weight of the surfactant Alkamuls® RC, marketed by Rhodia

and, as a solvent, the remainder of the compound of the examples.

Examples 2 are comparative examples where the product Rhodiasolv® ADMA10, or Rhodiasolv® ADMA810, product is used as a solvent, from Rhodia (Asia Pacific area): alkyldimethylamide solvents.
The following tests are conducted:

Visual observation at 25° C.—The aspect of the formulation is noted and the presence of crystals is optionally located

Visual observation at 0° C.—The formulation is placed for 7 days at 0° C. and the aspect of the formulation is noted and the presence of crystals is optionally located (test CIPAC MT39)

Visual observation at 0° C. with nucleation: A crystal of active material is introduced into the formulation having spent 7 days at 0° C. formulation, and the formulation is again placed for 7 days at 0° C. The aspect of the formulation is noted and the presence of crystals is optionally located.

| | | Aspect | Aspect | Aspect at |
|---|---|---|---|---|
| | | at | at | 0° C. with |
| Solvent | Active ingredient | 25° C. | 0° C. | nucleation |
| Rhodiasolv ® | Chlorpyrifos 40% | Limpid | Limpid | Limpid |
| ADMA 10 | | | | |
| Rhodiasolv ® | α-Cypermethrin | Limpid | Limpid | Limpid |
| ADMA 10 | 10% | | | |
| Rhodiasolv ® | Phenmedipham | Limpid | Limpid | Limpid |
| ADMA 10 | 16% | | | |
| Rhodiasolv ® | Propanil 36% | Limpid | Limpid | Limpid |
| ADMA 10 | | | | |
| Rhodiasolv ® | Tebuconazole 25% | Limpid | Limpid | Limpid |
| ADMA 10 | | | | |
| Rhodiasolv ® | Trifluralin 40% | Limpid | Limpid | Crystals |
| ADMA 10 | | | | |
| Rhodiasolv ® | Difenconazole 25% | Limpid | Limpid | Crystals |
| ADMA 10 | | | | |
| Rhodiasolv ® | Dimethoate 40% | Cloudy | Cloudy | Crystals |
| ADMA 10 | | | | |
| Rhodiasolv ® | Oxyfluorfen 22% | Limpid | Limpid | Crystals |
| ADMA 10 | | | | |
| Rhodiasolv ® | Propoxur 20% | Limpid | Limpid | Crystals |
| ADMA 10 | | | | |
| Rhodiasolv ® | Chlorpyrifos 40% | Limpid | Limpid | Limpid |
| ADMA 810 | | | | |
| Rhodiasolv ® | α-Cypermethrin | Limpid | Limpid | Limpid |
| ADMA 810 | 10% | | | |
| Rhodiasolv ® | Phenmedipham | Limpid | Limpid | Limpid |
| ADMA 810 | 16% | | | |
| Rhodiasolv ® | Propanil 36% | Limpid | Limpid | Limpid |
| ADMA 810 | | | | |
| Rhodiasolv ® | Tebuconazole 25% | Limpid | Limpid | Limpid |
| ADMA 810 | | | | |
| Rhodiasolv ® | Azoxystrobin 25% | Not | Not | Not soluble |
| ADMA 810 | | soluble | soluble | |
| Compound 1 | Chlorpyrifos 40% | Limpid | Limpid | Limpid |
| Compound 1 | α-Cypermethrin | Limpid | Limpid | Limpid |
| | 10% | | | |
| Compound 1 | Phenmedipham | Limpid | Limpid | Limpid |
| | 16% | | | |
| Compound 1 | Propanil 36% | Limpid | Limpid | Limpid |
| Compound 1 | Tebuconazole 25% | Limpid | Limpid | Limpid |
| Compound 1 | Trifluralin 40% | Limpid | Limpid | Limpid |
| Compound 1 | Difenconazole 25% | Limpid | Limpid | Limpid |
| Compound 1 | Dimethoate 40% | Limpid | Limpid | Limpid |
| Compound 1 | Oxyfluorfen 22% | Limpid | Limpid | Limpid |
| Compound 1 | Propoxur 20% | Limpid | Limpid | Limpid |
| Compound 1 | Azoxystrobin 25% | Limpid | Limpid | Crystals |
| Compound 4 | Phenmedipham | Limpid | Limpid | Limpid |
| | 16% | | | |
| Compound 4 | Propanil 36% | Limpid | Limpid | Limpid |
| Compound 4 | Tebuconazole 25% | Limpid | Limpid | Limpid |
| Compound 4 | Trifluralin 40% | Limpid | Limpid | Crystals |
| Compound 4 | Difenconazole 25% | Limpid | Limpid | Limpid |
| Compound 4 | Dimethoate 40% | Limpid | Limpid | Limpid |
| Compound 4 | Oxyfluorfen 22% | Cloudy | Cloudy | Cloudy |
| Compound 4 | Propoxur 20% | Limpid | Limpid | Limpid |
| Compound 4 | Azoxystrobin 25% | Crystals | Crystals | Crystals |

1. A phytosanitary formulation comprising at least:
a) an active phytosanitary product,
b) a compound of the following formula (I):
R^{a}R^{b}C(OR¹)-A-C(O)—NR²R³ (I)
wherein:
R^{a} and R^{b}, identical or different, are groups selected from a hydrogen atom and linear or branched alkyl groups;
R¹ is a saturated or unsaturated, linear or branched hydrocarbon group comprising an average number of carbon atoms ranging from 1 to 36, optionally cyclic, optionally aromatic, optionally substituted
R² and R³, either identical or different, are groups selected from a hydrogen atom and saturated or unsaturated, linear or branched hydrocarbon groups comprising an average number of carbon atoms ranging from 1 to 36, optionally cyclic, optionally aromatic, optionally substituted, R² and R³ may optionally form together a ring comprising the nitrogen atom to which they are bound, optionally substituted and/or optionally comprising an additional heteroatom,
A represents a linear or branched alkyl group, the main chain of which comprises at least 2 carbon atoms;
c) optionally a surfactant, and
d) optionally water.

2. The formulation according to claim 1, comprising a compound of formula (I) wherein R^{a} and R^{b}, identical or different, are groups selected from a hydrogen atom and linear or branched C₁-C₃ alkyl groups.

3. The formulation according to claim 1, comprising a compound of formula (I) wherein at least one of the groups selected from R^{a} and R^{b} is a group selected from linear or branched C₁-C₆ alkyl groups.

4. The formulation according to claim 1, comprising a compound of formula (I) wherein A represents a linear or branched alkyl group, the main chain of which comprises from 2 to 8 carbon atoms.

5. The formulation according to claim 1, comprising a compound of formula (I) wherein R² and R³, identical or different, are selected from methyl, ethyl, propyl(n-propyl), isopropyl, n-butyl, isobutyl, n-pentyl, amyl, isoamyl, hexyl, cyclohexyl groups, mixtures and/or combinations thereof, R² and R³ may also be such as to form together with the nitrogen atom a morpholine, pyrrolidine piperazine or piperidine group.

6. The formulation according to claim 1, comprising a compound of formula (I) wherein R¹ is a hydrocarbon group selected from methyl, ethyl, propyl(n-propyl), isopropyl, n-butyl, isobutyl, n-pentyl, amyl, isoamyl, hexyl, or cyclohexyl groups.

7. The formulation according to claim 1, comprising a compound of formula (I) selected from one of the following compounds:

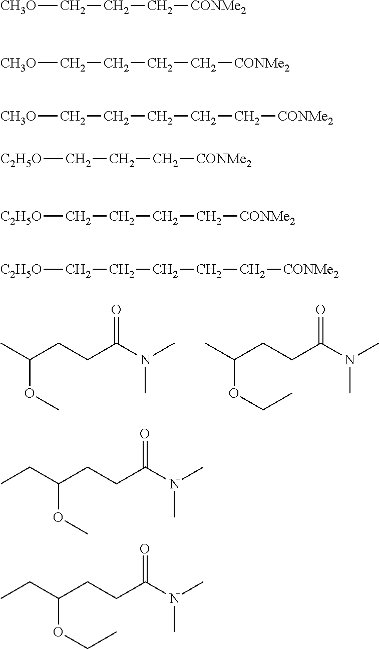

8. The formulation according to claim 1, wherein the formulation comprises a surfactant c), selected from non-ionic surfactants, anionic surfactants in a salified or acid form, non-ionic polyalkoxylated, cationic surfactants or amphoteric surfactants.

9. The formulation according to claim 1, wherein the formulation is in the form of an emulsifiable concentrate (EC), of a liquid concentrate (SL), of a concentrated emulsion (EW), of a suspo-emulsion (SE), or of a micro-emulsion (ME).

10. A solvent, co-solvent, crystallization inhibitor, and/or an agent for increasing bioactivity of an active phytosanitary product in phytosanitary formulations comprising a) said active phytosanitary product, c) optionally a surfactant, and d) optionally water, comprising a compound of formula (I)
R^{a}R^{b}C(OR¹)-A-C(O)—NR²R³ (I)
wherein:
R^{a} and R^{b}, identical or different, are groups selected from a hydrogen atom and linear or branched alkyl groups;
R¹ is a saturated or unsaturated, linear or branched hydrocarbon group comprising an average number of carbon atoms ranging from 1 to 36, optionally cyclic, optionally aromatic, optionally substituted
R² and R³, either identical or different, are groups selected from a hydrogen atom and saturated or unsaturated, linear or branched hydrocarbon groups comprising an average number of carbon atoms ranging from 1 to 36, optionally cyclic, optionally aromatic, optionally substituted, R² and R³ may optionally form together a ring comprising the nitrogen atom to which they are bound, optionally substituted and/or optionally comprising an additional heteroatom,
A represents a linear or branched alkyl group, the main chain of which comprises at least 2 carbon atoms.
